(19) United States
(12) Patent Application Publication
(10) Pub. No.: US 2003/0113352 A1
(43) Pub. Date: Jun. 19, 2003

(54) Neutralization of stratum corneum to treat hyperkeratotic skin lesions

(75) Inventors: Peter M. Elias, Mill Valley, CA (US); Kenneth R. Feingold, San Rafael, CA (US); Joachim W. Fluhr, San Francisco, CA (US); Theodora M. Mauro, San Francisco, CA (US); Martin J. Behne, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/017,841

(22) Filed: Dec. 14, 2001

(51) Int. Cl. A61K 31/4965

(52) U.S. Cl. 424/401; 514/255.06

(57) ABSTRACT

Hyperkeratotic skin lesions are treated by the topical administration of inhibitors of hydrogen ion pumps, transporters, and antiporters, or buffers with a pH of 7.0 or greater, and inhibitors of physiological processes that generate free fatty acids from complex lipids.

### STATEMENT AS TO RIGHTS TO INVENTIONS MADE UNDER FEDERALLY SPONSORED RESEARCH AND DEVELOPMENT

This invention was made at least in part with assistance from the United States Federal Government, under Grant No. AR 19098 of the National Institutes of Health. As a result, the government has certain rights in this invention.

This invention resides in the technical field of topical formulations for application to skin and mucous membranes and to the treatment of subjects suffering from skin diseases or disorders involving hyperkeratosis (increased skin thickness or excessive intercellular cohesion of the stratum corneum) and of subjects suffering from conditions that can be improved by the transdermal delivery of drugs or nutrients or both.

### BACKGROUND OF THE INVENTION

Mammalian epidermis consists of a continuously self-replicating, stratified, keratinized squamous epithelium, the principal cells of which are keratinocytes. The population of keratinocytes undergoes continuous renewal throughout life. A mitotic layer of basal cells at the base replaces cells at the surface as they slough off. As they move above the basal layer of the epidermis, keratinocytes undergo a process of differentiation known as keratinization. They undergo progressive changes in shape and content, eventually transforming from polygonal living cells, into anucleate, non-viable, flattened squames replete with keratin and other proteins. The stratum corneum is the outermost layer of the epidermis and the final product of epidermal differentiation.

The stratum corneum of mammalian skin displays a strongly acidic pH. The pH of the upper stratum corneum measures approximately 4.5-5.0 while the pH of the lower stratum corneum (above the outermost nucleated granular cell layer) approaches neutrality. Thus, the stratum corneum experiences a pH differential of more than two pH units over a vertical space of less than 100 microns. This pH gradient occurs not only in human but also in rodent skin despite its much thinner stratum corneum.

Although acidity is thought to be essential for certain stratum corneum functions, the acid mantle of the stratum corneum is believed to originate as a secondary consequence of biological functions intrinsic to the epidermis; the “acid mantle” is not seen as being generated by the cells of the stratum corneum itself. First recognized decades ago (Schade, H., et al., Klin. Wochenschr. 7:12-14 (1928)), understanding of the origin and function of the “acid mantle” of the stratum corneum is still incomplete. Nonetheless, it is known that the activities of several proteases that are important for desmosomal cleavage are modulated by pH, functioning optimally at neutral pH. Whatever the nature of its origin, the acid mantle of the stratum corneum is an important factor in maintaining the delicate homeostasis of the epidermis.

The mammalian epidermis forms a self-renewing and self-repairing interface between the body and the environment, and is a major site of intercommunication in both directions between the two. Within limits, it forms an effective barrier against microbial invasion and also functions as a permeability barrier to guard against excessive transcutaneous water loss. The skin protects against osmotic damage and is selectively permeable to a variety of chemical substances. In addition, the skin protects against mechanical, thermal, chemical and photic damage.

In functioning to protect the organism from insults and damage inflicted by rubbing, pressure, inflammation or the radiation of sunlight, the epidermis responds with local thickening (hyperkeratosis). This normal response of the epidermis to its environment can lead to a number of skin disorders that range from cosmetic nuisances such as calluses or seborrheic keratoses, to painful or even troublesome medical conditions such as cancer. Treatment of such conditions must be undertaken with care as the skin is an important social and sensory organ.

Clearly a need exists for the treatment and /or removal of hyperkeratotic skin lesions in all their various forms and degrees of severity. In addition, manipulating the barrier function of normal stratum corneum for the purpose of administering drugs or nutrients transcutaneously could improve treatments and therapies for a wide range of conditions or diseases.

### SUMMARY OF THE INVENTION

It has now been discovered that inhibition or deletion of the NHE1 sodium/ hydrogen antiporter is associated with an increase of the pH within the stratum corneum and that this pH increase leads to reduced stratum corneum cohesion, compromised integrity and impaired barrier function. The reduction in stratum corneum cohesion and integrity is useful for treating or lessening the symptoms of hyperkeratosis by making it easier to detach excess stratum corneum as necessary. Diminishment of barrier function is useful for enhancing the transdermal delivery of drugs and nutrients. In accordance with this invention, therefore, the NHE1 antiporter is regulated to increase the pH of the stratum corneum toward neutrality by the application of inhibitors of NHE1.

It has also been discovered that applications of phospholipase inhibitors increase the pH of the stratum corneum toward neutrality and thereby reduce stratum corneum cohesion and integrity and hinder barrier function. Accordingly, this invention further resides in the topical administration of phospholipase inhibitors to achieve neutralization of stratum corneum pH, weaken cohesion and integrity of the stratum corneum and impair barrier function.

The invention also resides in the application of inhibitors of processes that generate free fatty acids from complex lipids.

This invention further resides in the application of inhibitors of processes that lead to the generation of urocanic acid or other organic acids.

The invention also resides in the application of buffers with a pH greater than 7.0.

The invention therefore resides in the topical application of substances that raise the pH of the stratum corneum toward neutrality with the purpose of weakening its cohesion and integrity and disrupting barrier function. Some of the applied substances are inhibitors of hydrogen ion pumps, transporters, and antiporters, in particular, inhibitors of the NHE1 sodium/proton antiporter. Other substances of this invention are buffers with a pH of 7.0 or greater, inhibitors of processes that generate free fatty acids from complex lipids especially inhibitors of phospholipases, and inhibitors of processes that generate urocanic acid and other organic acids.

Other features, embodiments and advantages of the invention will become apparent from the description that follows.

### BRIEF DESCRIPTION OF THE DRAWINGS

FIG. 1 is a plot of pH vs. time measured ratiometrically as the fluorescence at 530 nm from cells labeled with the pH sensitive dye BCECF. Activity of the NHE antiporter was tested by measuring the cell's ability to recover from an acid load. The plot shows pH (x-axis) vs. time (y-axis, seconds). (A) starting equilibration. Arrow indicates the addition of NH₄Cl, followed by a spike of alkalinization and after dissociation of the NH₄⁺ ion, an acid load inside the cells. Cells slowly extrude H⁺ reestablishing a more neutral pH (B). The addition of amiloride at 1 μM (C) blocks the recovery from an identical acid load. (D) The wash out period shows recovery from the acid load indicating a non-permanent, non-toxic effect of amiloride.

FIG. 2 is a bar graph showing the changes in pH of the stratum corneum after skin is tape stripped then exposed to acidic (pH 5.5) or neutral (pH 7.4) buffers with or without NHE 1 inhibitors.

### DETAILED DESCRIPTION OF THE INVENTION AND SPECIFIC EMBODIMENTS

The acidic pH environment of the stratum corneum is important for epidermal biology. Functions such as stratum corneum cohesion and desquamation, and the formation and maintenance of an epidermal permeability barrier all depend on the pH gradient.

It has now been discovered through its action in the hydrolysis of phospholipids, phospholipase is critical for the formation and maintenance of the acidic pH gradient of the stratum corneum. Phospholipase contributes to the generation and maintenance of the acid pH environment of the epidermis through the production of fatty acids which result from the hydrolysis of its phospholipid substrates. According to one aspect of this invention, therefore, a phospholipase inhibitor is applied to the epidermis in an amount that is effective at reducing the integrity and cohesion of the stratum corneum and lessening barrier function. Preferred phospholipase inhibitors are those that inhibit the activity of phospholipase A₁ and phospholipase A₂ such as Bromphenacyl bromide (BPB), 1-hexadecyl-3-trifluoroethylglycero-sn-2-phosphomethanol (MJ33), quinacrine and mepacrine. Most preferred are inhibitors of natural or bioengineered 14 kDA type 1 secretory phospholipase A₂ and secretory phospholipase A₂ pancreatic type such as bromphenacyl bromide (BPB) and 1-hexadecyl-3-trifluoroethylglycero-sn-2-phosphomethanol (MJ33).

It has also been discovered that that the application of free fatty acids will acidify the pH of the stratum corneum and protect its integrity and cohesion. Therefore, according to one aspect of this invention, an inhibitor of any process that generates free fatty acids from complex lipids is applied in an amount that is effective at raising the pH of the stratum corneum toward neutrality thereby reducing stratum corneum cohesion and integrity and diminishing barrier function. In a preferred embodiment, the inhibitor is an inhibitor of β-glucocerebrosidase. In a particularly preferred embodiment, the inhibitor is bromconduritol-β-epoxide (BrCBE).

The pH of the epidermis is also raised toward neutrality in accordance with this invention by the administration of substances that inhibit the generation of urocanic acid and other organic acids. Preferred inhibitory substances are histidase inhibitors.

It has also been discovered that the sodium/hydrogen antiporter, NHE1, is located in the outer differentiated layers of the epidermis and that its activity contributes significantly to acidification of the stratum corneum. According to one aspect of this invention, activity of the sodium/hydrogen antiporter, NHE1, is blocked by the application to the epidermis of an inhibitor of NHE1 in amounts that are effective at raising the pH of the stratum corneum toward neutrality, thereby reducing cohesion, compromising integrity and diminishing barrier function of the stratum corneum. Preferred inhibitors of the NHE1 antiporter are members selected from the group consisting of amiloride, dimethylamiloride, ethylisopropylamiloride, methylpropylamiloride, methylisobutylamiloride, hexametyleneamiloride, HOE694 and HOE642. Most preferred NHE1 inhibitors are amiloride and HOE694.

In another aspect of this invention, buffers with a pH of 7.0 or greater are applied to the epidermis in amounts that are effective for raising the pH of the stratum corneum toward neutrality thereby decreasing the cohesion and integrity of the stratum corneum and hindering barrier function. Examples of such buffers are HEPES-based buffers (where HEPES is N-[2-hydroxyethyl]piperazine-N′-[2-ethanesulfonic acid]), MES-based buffers (where MES is 2-(N-morpholino)ethanesulfonic acid), MOPS-based buffers (where MOPS is 3-(N-morpholino)propanesulfonic acid), PIPES-based buffers (where PIPES is 1,4-piperazinediethanesulfonic acid), TES-based buffers (where TES is N-tris[hydroxymethyl]methyl-2-aminoethanesulfonic acid), phosphate based buffers, citrate based buffers and bicarbonate based buffers. HEPES-based buffers are particularly preferred. The pH of the buffer may vary, but best results will generally be achieved with a buffer pH in the range of 7.0-8.0. A particularly preferred buffer pH is 7.4.

This invention also extends to topical compositions that contain two or more active ingredients from among the various types and examples set forth above.

This invention is useful for the treatment of diseases or conditions that involve hyperkeratosis and excessive intercellular cohesion of the stratum corneum. The invention can be employed in order to facilitate the removal of excess pathological or normal stratum corneum. The invention is also useful for the manipulation of normal stratum corneum cohesion, integrity and barrier function in order to facilitate the transdermal delivery of co- or sequentially applied drugs or nutrients. Treatment or therapy or both can be achieved for example, by administration of:

Inhibitors of processes that lead to the generation of free fatty acids from complex lipids,

phospholipase inhibitors, preferably inhibitors of secretory phospholipase A₂,

inhibitors of hydrogen ion pumps, transporters or antiporters, preferably inhibitors of the NHE1 sodium-proton antiporter,

inhibitors of processes that lead to the generation of urocanic and other organic acids, or

neutral pH buffers.

Examples of conditions that are treatable by this invention are the removal of excess pathological stratum corneum and the removal of excess normal stratum corneum as in calluses, bunions and callosities.

In other embodiments, this invention resides in the administration of the agents listed above for the removal or debridement of hyperkeratotic growths and neoplasms, most notably seborrheic keratoses, warts and hypertrophic actinic keratosis.

In still other embodiments, this invention resides in the administration of the agents listed above for increased transdermal delivery of co- or sequentially applied drugs or nutrients for systemic, regional (e.g. underlying joints, fat, blood vessels, or hair bulb) and local (skin) applications.

In the practice of this invention, the neutralizing agents will be administered as active ingredients in a formulation that is pharmaceutically acceptable for topical administration. These formulations may or may not contain a vehicle, although the use of a vehicle is preferred. Preferred vehicles are non-lipid vehicles, particularly a water-miscible liquid or mixture of liquids. Examples are glycerol, urea, methanol, ethanol, isopropanol, ethylene glycol, propylene glycol, and butylene glycol, and mixtures of two or more of these compounds.

The concentration of active ingredient in the vehicle is not critical to this invention and may vary widely while still achieving a therapeutic effect, a preventive effect, or both. In most cases, the concentration will fall within the range of from about 10 μM to about 1,000 μM, although for certain active ingredients, the concentration may vary outside this range. For example, the preferred concentrations of some active ingredients will be within the range of about 10 μM to about 1,000 μM, and in others the preferred range will be 100 μM to about 1,000 μM.

The invention is generally applicable to the treatment of the skin of terrestrial mammals, including for example humans, domestic pets, and livestock. The invention is of particular interest in treating humans for the conditions described above or for preventing these conditions from becoming manifest.

### EXAMPLES

The following examples are offered to illustrate, but not to limit the claimed invention.

Materials and Methods for Examples 1 through 10

Animals: Male hairless mice (Skh1/Hr, Charles River Laboratories, Wilmington, Mass.), 8-12 weeks old, were fed Purina mouse diet and water ad libitum. Heterozygous, NHE1 deficient mice (Bell et. al., 1999) were bred in house, from founders received from Dr. G. E. Shull, Cincinnati, Ohio. Each litter was genotyped separately. Functional experiments were performed on animals aged 6-10 weeks.

Chemicals: Propylene glycol, ethanol, NaOH and HCL were from Fischer Scientific. Amiloride (N-Amido-3,5-diamino-6-chloropyrazinecarboxamide), bromphenacyl bromide (BPB), HEPES (N-[2-hydroxyethyl]piperazine-N′-[2-ethanesulfonic acid]) and TES (-tris[hydroxymethyl]methyl-2-aminoethanesulfonic acid) were purchased from Sigma Chemical Co. Bio-Rad Protein Reagent, Bradford protein assay kits, lyophilized bovine plasma gammaglobulin (GG) and Bovine Serum Albumin were purchased from Bio-Rad Laboratories. 2′,7′-Bis(carboxyethyl)-5,6-carboxyflourescein-AM ester (BCECF-AM) was from Molecular Probes (Eugene, Oreg.). HOE694 (3-methylsulfonyl-4-piperidino-benzoylguanidinemethanesulfonate) was kindly provided by Dr. H. J. Lang, Hoechst-Marion-Roussel AG, Frankfurt, Germany. 1-Hexadecyl-3-triflouroethylglycero-sn-2-phosphomethanol (MJ33) was synthesized as described previously (Jain, M. K., et al., Biochemistry 30:10256-68 (1991)). The stripping for the protein assay was performed with 22 mm D-Squame 100 tapes purchased from CuDerm (Dallas, Tex.).

Permeability Barrier Studies: Normal hairless mice were treated topically twice a daily for three days with BPB (4 mg/mL) or MJ33 (4 mg/mL) both in propylene glycol:ethanol (7:3 v/v) vehicle, or the vehicle alone on an area of 5-6 cm on the backs and flanks as described previously (Mao-Qiang, M., et al., J. Lipid Res. 36:1926-35 (1995)). The doses employed were shown previously to be non-toxic to Murine skin (Mao-Qiang, M., et al. (1995); Mao-Qiang, M., et al., J. Invest. Dermatol. 106:57-63 (1996)), and to inhibit secretory phospholipase A₂ (sPLA₂) activity selectively in different cell types (Gelb, M. H., et al., Faseb J. 8:916-24 (1994); Jain, M. K., et al. (1991)). The acidification experiments were performed with 10 mM HEPES-buffer, adjusted to either pH 5.5 or pH 7.4 as follows: One flank of anesthetized mice was immersed on a mesh netting, as described previously (Lee, S. H., et al., J. Clin. Invest. 89:530-8 (1992)). The mice were anesthetized with chloral hydrate (Morton Grove Pharmaceuticals, Morton Grove, Ill., USA). After 3 hours immersion at 37° C., the mice were removed, and the remaining buffer was gently blotted off. After 15 minutes, barrier function was determined by measurement of transepidermal water loss(TEWL) with an electrolytic water analyzer (MEECO®, Warrington, Pa.). Surface pH was measured with a flat, glass surface electrode from Mettler-Toledo (Giessen, Germany), attached to a pH meter (Skin pH Meter PH 900, Courage& Khazaka, Cologne, Germany). Individually tested sites were covered with Hilltop Chambers (HtC, nominal volume 200 mL), which were reapplied following each indvidual measurement. For topical applications, solutions of HEPES buffer (10 mM) at either pH 7.4 or pH 5.5, contained either amiloride (5 mM), HOE694 (7.5 mM), or buffer alone. The inhibitor-concentrations represented the published 50% inhibitory concentrations (IC₅₀, values in mM) for the NHE 1 inhibitory compounds in fibroblasts.

The stratum corneum was removed by several strippings with adhesive tape (Tesatape, Beiersdorf, Germany) inducing an increase in TEWL levels above baseline (from 2-5 to approximately 10-15 g/m²/h). Two sites were prepared on each animal, and TEWL and surface pH were measured at 0, 2, 5 and 24 hrs following the stripping and the applications of inhibitors. Biopsies were taken for electron microscopy from treated and control sites, at 5 and 24 hours. For studies in the NHE1 knockout mice, homozygous (−/−) mice were compared to their wildtype (+/+) littermates. Flanks of these mice were shaved, and barrier homostasis was studied 48 hours later. For tape stipping of these animals, D-Squame disks (Cu-derm, Dallas, Tex.) were used, as Tesatape was too disruptive for application to shaved, hairy mouse skin.

Cell Culture and Western Immunoblotting: Second passage cultured human Keratinocytes from human foreskin (CHK) were grown in low calcium medium (0.03 mM Ca²⁺, Cascade 154, Cascade Biologics, Eugene, Oreg.) until they reached approximately 60% confluence. Cells were then incubated with various concentrations of HOE694, and compared to cell grown in high calcium medium (1 mM or 2 mM Cascade 154) with HOE694. Following incubations of 48 hours, CHK were harvested and frozen in liquid nitrogen

The cells were thawed, homogenized by sonication. Their protein content determined, and gels were loaded with equal amounts per sample and lane. Western Immunoblotting was performed using 7.5% SDS-PAGE, as described previously (Laemmli, 1970). Following transfer of protein to PVDF membranes, blots were incubated overnight with primary antibody at 4° C. (monoclonal anti-INV, clone SY5, Sigma Immunochemicals, St Louis, Mo.). Secondary antibody was applied and blots were incubated at room temperature for 2 hours(peroxidase conjugated anti mouse; Amersham PharmaciaBiotech Inc., Piscataway, N.J.). Final detection was performed by chemiluminescence (ECL kit; Amersham). NHE1 was detected in membrane fractions prepared from CHK (cultured as follows: undifferentiated keratinocytes were cultured with 0.03 mM calcium until reaching ˜80% confluence; differentiated cells were maintained in 1.2 mM until reaching either 4 or 7 days post confluence). The primary antibody used was mouse monoclonal anti-NHE1 (Chemicon Int., Temecula, Calif.). For AB/AG competition studies, the primary antibody was preabsorbed with the peptide used for creating the antibody (Alpha Diagnostic, San Antonio, Tex.).

Ultrastructural methods: Freshly obtained biopsies from mouse skin were fixed directly in modified Karnovsky's fixative, postfixed with reduced osmium tetroxide (OSO₄) and then imbedded in an Epon-epoxy mixture. For visualization of lipid-enriched, lamellar bilayer structures, some samples were post fixed in ruthenium tetroxide (RuO₄). Sections were cut on a Reichert Ultracut E microtome, counterstained with uranyl acetate and lead citrate, and viewed in a Zeiss 10 CR electron microscope, operated at 60 kV.

Immunohistochemistry: Fresh samples of normal human skin from surgical margins, or biopsies from NHE1 +/+ or −/− mice were formaldehyde fixed, parafin embedded, and sectioned (5 μm). For immunolabeling of NHE1, a rabbit polyclonal antibody was used (Chemicon Int., Temecula, Calif.), which was detected via FITC-labled, secondary goat anti-rabbit antibody (Cappel, Organanon Teknika Corp., Durham, N.C.). Sections were counterstained with propidium iodide (Sigma, St. Louis, Mo.), and pictures were taken on a Leica TCS-SP confocal microscope.

Immunofluorescence Staining: Hairless mouse skin was excised with a 6 mm punch biopsy, and the subcutaneous fat was removed. Tissue sections were incubated for 1 hour in blocking buffer (1% bovine serum albumin, 0.1% cold water fish gelatin in phosphate buffered saline (PBS)), and were then incubated for 1 hour further at room temperature with 1:500 dilution of polyclonal rabbit anti-mouse desmoglein 1 antibody (gift of Dr. John Stanley, University of Pennsylvania) diluted in blocking buffer. The tissue was then washed with blocking buffer and incubated one hour at room temperature with fluoroscein, isothiocyanate-conjugated, goat anti-rabbit IgG antibody (DAKO, Carpinteria, Calif., USA), diluted in blocking buffer. Either preincubation of DSG 1 antibody with DSG 1-recombinant protein (gift of Dr. Masayuki Amagai, Keio University, Tokyo, Japan), or omission of the DSG 1 primary antibody eliminated specific staining. Tissue sections then were washed with PBS and coverslipped before visualization with confocal microscope (Leica TCS SP, Heidelberg, Germany) using FITC at an excitation wavelength of 494 nm and an emisson wavelength of 518 nm.

Protein Assay on Sequential Tape Strips: The protein assay utilized the Bradford dye-binding procedure for quantification of total protein (Bradford, 1976). HEPES buffer and propylene glycol:ethanol, the two vehicles used in these studies, are known to be compatible with this assay. Before stripping the stratum corneum, the skin surface was cleaned with a single ethanol wipe. D-Squame tapes then were placed sequentially to the test areas for about 3 sec each, removed with forceps, and stored in glass scintillation vials at 5° C.

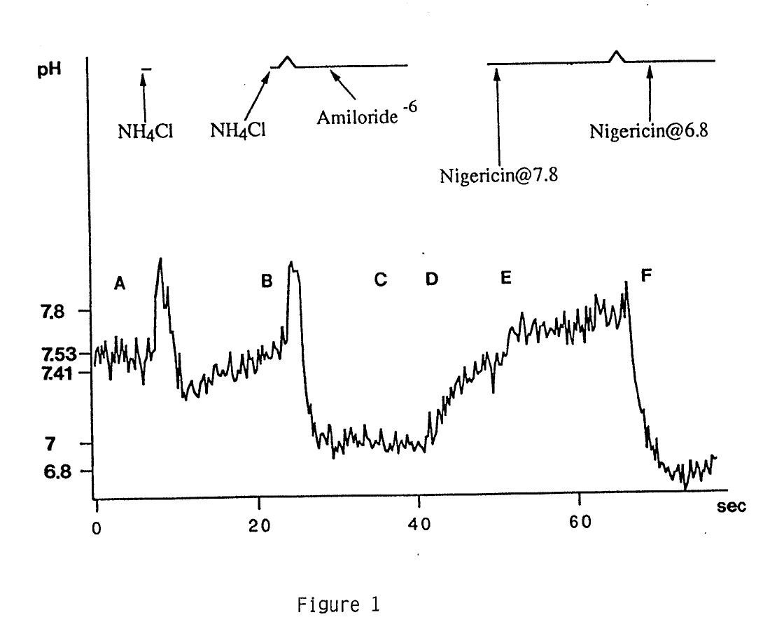

The amount of protein removed per D-Squame was measured, by a modification of the method of Dreher, F., et al., Acta Derm. Venereol 78:186-9 (1998). The microassay system was shown to be linear in the range of 1-10 μg/mL, using human stratum corneum removed from a heel callosity (FIG. 1a). The protein content per stripping was determined with the Bio-Rad protein assay kit. Lyophilized, bovine gammaglobulin (GG) was used as standard in all assays, because it correlated best with human stratum corneum. Each tape was incubated with 1 M NaOH for 1 hour at 37° C. in an incubator shaker at 80 rpm, and neutralized thereafter with 1 mL of 1 M HCl in the scintillation vials. Subsequently, 0.2 mL of this solution was incubated in 0.6 ml distilled water plus 0.2 mL of the Bio-Rad protein dye for 5 minutes in borosililate tubes. After incubations, the reagents were transferred to polystyrene cuvettes, and absorption was measured with a Genesys 5 spectrophotometer (Spectronic, Rochester, N.Y.) at 595 nm. An empty D-Squame tape, as well as distilled water incubated with Bio-Rad dye, served as negative controls. The amount of calculated protein was then normalized to skin surface area (μg/cm²). The amount of removed protein per D-Squame strip agreed with previous reports in untreated skin of hairless mice (i.e., range of 2.5-4 μg/strip) (Weber, S.U., et al., J. Invest. Dermatol. 113:1128-32 (1999)).

Assessment of Intracellular pH: Keratinocytes were plated on glass coverslips and grown in Cascade 154 medium containing 0.07 mM Ca²⁺. The coverslips were incubated in 145 μM BCECF-AM ester at room temperature for 5 minutes, then rinsed for 30 minutes in buffer containing either 28 mM HEPES, 136 mM NaCl, 5 mM KCl, 0.07 mM CaCl₂ and 10 mM glucose, pH 7.4 or Ringer solution (136 mM NaCl, 5 mM KCl, 0.03 mM Ca2+, 28 mM TES, 14 mM Na HCO₃, NaOH to adjust the pH to 7.4, and 10 mM glucose (pH 7.4, 330 mOsm)). BCECF-AM-ester is membrane permeable; once inside cells it is de esterified by endogenous carbonic anhydrase, trapping flourescently-active, acidic BCECF inside the cells. The BCECF-loaded cells were placed in a superfusion chamber mounted on a inverted microscope (Nikon). The keratinocytes were superfused with one of the two solutions, and baseline intracellular pH was monitored constantly using a ratiometric method (Paradiio et al (1987) Am J. Physiol. 253:C30-6). Briefly, BCECF loaded cells were alternately illuminated every 10 seconds with brief flashes (200 milliseconds) of 440 nm and 490 nm light. The resultant fluorescence (at 530 nm) from each stimulating wavelength was measured with a CCD camera (Hamamatsu) digitally ratioed, and stored using a software program (Fluor, Universal Imaging Co., West Chester, Pa.). Whereas fluorescence emission at 440 nm excitation remains constant, indicative of dye concentration, emission from 490 nm varies proportionally with changes of intracellular pH. By ratioing the two signals, intracellular pH is measured as a brightness signal corrected for the intracellular dye concentration.

After equilibration under basal conditions , cells were either alkalinized or acidified by superfusion for two minutes with Ringer solution in which 20 mM NH₄Cl had been substituted for NaCl (i.e. 116 mM NaCl, 20 mM NH₄Cl), amiloride (μM) then was added to the bath solution. Calibration was performed at the end of each experiment by permeablizing the cells to protons with nigericin, thereby equilibrating intra- and extracellular pH. Superfusing the cells with solutions containing 85 mM NaCl, 50 mM KCl, 0.07 mM CaCl₂, 10 mM nigericin (a K+/H+ exchanger), and either 28 mM HEPES, or 28 mM TES bubbled with 5% CO₂, pH 6.8 or 7.8, allowed to calibrate intracellular signals.

mRNA Measurements by RT-PCR: Total RNA was prepared using the Qiagen RNeasy method (Qiagen, Valencia, Calif.) from both second passage CHK (grown in 1.2 mM Ca²⁺ to 4 days post confluence) or from human skin (from normal surgical margins) incubated with dispase (50 U/mL, Gibco, Life Technologies, Rockville, Md.) to prepare whole epidermis. RNA from each sample was reverse transcribed using Gibco reverse transcriptase. The cDNA was then amplified by PCR, employing the following primer set for human NHE1

5′ ACC CTG CTC TTC TGC CTC ATC G3′
5′ CCT GCT TCA TCT CCA TCT TGT G3′

The PCR product was separated on an agarose gel, purified, and subcloned into one shot competent cells (InVitrogen, Carlsbad, Calif.), plasmid DNA was prepared and sequenced. As a negative control, the same amplification without prior reverse transcription, gave no transcription product.

Statistical Analysis: Statistical analyses were performed using Prism 3 (GraphPad Software Inc., San Diego, Calif.). Normal distribution was tested before calculating the comparison. In the three group comparisons an ANOVA was first calculated followed by a post-hoc test (Bonferroni). Two groups were compared with an unpaired t-test.

### Example 1

The experiments reported in this example demonstrate that inhibition of secretory phospholipase A₂ results in an increase in stratum corneum pH and that this pH increase is associated with diminished barrier function, compromised integrity and reduced cohesion of the stratum corneum.

Male hairless mice 8-12 weeks old were treated twice daily for three days with topical applications of the secretory phospholipase A₂ inhibitor bromphenacyl bromide (BPB) at a concentration of 4 mg/mL in propylene glycol:ethanol vehicle (7:3 v/v), or the vehicle alone, on an area of 5-6 cm on their backs and flanks, as described in materials and methods. After one day of treatment, the pH of the skin of the treated hairless mice increased significantly from a starting value of about pH 5.6 to nearly pH 6.0. Over the three day trial period, the pH of the BPB treated skin sites continued to show an increase relative to that of vehicle treated sites; the final average pH for treated sites on day 3 of the trial was nearly pH 6.4, whereas that of vehicle treated sites was close to pH 5.4.

By day two of the experiment, daily topical applications of secretory phospholipase A₂ inhibitor produced an abnormality in barrier function of the treated skin as measured by rates of transepidermal water loss (TEWL). TEWL levels for the treated sites were near 6 g/m²/h, whereas the vehicle treated sites showed TEWL levels closer to 2.5 g/m²/h. By day three of the experiment, TEWL rates for the treated sites were increased to nearly 11 g/m²/h. In contrast the vehicle treated sites had TEWL rates near 3 g/m²/h.

The integrity of the stratum corneum, evaluated as the number of tape strippings required to produce elevated TEWL levels, was markedly abnormal after three days of BPB treatment. A significant abnormality was present by the second stripping, and integrity continued to decline thereafter. By the second strip, sites treated with BPB had TEWL levels close to 20 g/m²/h, whereas vehicle treated sites had rates closer to 5 g/m²/h. This abnormality persisted and was amplified over the course of the experiment. By the fifth and final strip, TEWL rates were near 90 g/m²/h for the BPB treated sites and close to 25 g/m²/h for the vehicle treated sites.

The decline in integrity was paralleled by progressive loss of stratum corneum cohesion, as measured by quantification of the amount of protein removed per D-Squame stripping. As was the case for pH increase, barrier function (measured as TEWL) and integrity, cohesion of the stratum corneum was impaired by the application of secretory phospholipase A₂ inhibitors. The amount of protein removed per D-Squame stripping was significantly greater from the experimental sites than the vehicle treated sites, even by the first strip; vehicle treated sites showed protein losses of about 5 μg/cm², whereas the losses from BPB treated sites were close to 20 μg/cm². This trend continued with further stripping. After five strips the vehicle treated sites had lost about 20 μg protein/cm² of surface stripped, where the BPB treated sites lost about 85-90 μg/cm². Thus, applications of the secretory phospholipase A₂ inhibitor, BPB, result in an increased skin surface pH, and this pH increase is accompanied by altered barrier function and reduced stratum corneum integrity and cohesion.

Because BPB is an alkylating agent, and could non-specifically affect other cellular processes, additional studies were performed using a chemically unrelated competitive inhibitor of secretory phospholipase A₂, 1-Hexadecyl-3-triflouroethylglycero-sn-2-phosphomethanol (MJ33). MJ33 is a highly specific inhibitor of group 1 secretory phospholipase A₂. Like BPB, MJ33 produced an increase in stratum corneum surface pH vs vehicle alone (5.87+/−0.06 vs 5.60+/−0.05; p=0.0023). Moreover, repeated applications of MJ33 produced a progressive abnormality in stratum corneum barrier function resulting in a 2-3 fold increase in TEWL rates by day three (5.19+/−.81 vs. 2.97 +/−16; p<0.001, for MJ33 vs. vehicle treated animals).

As with BPB treatments, three days of MJ33 applications also progressively reduced stratum corneum integrity and cohesion. By day 3 of the experiment, TEWL rates for the MJ33 treated sites approached 75 g/m²/hr after three consecutive tape strippings, while the vehicle treated sites showed TEWL rates closer to 40 g/m² hr. The reduced integrity of MJ33 treated sites was reflected by a parallel change in stratum corneum cohesion. By day 3 of the experiment, vehicle treated sites lost about 20 μg protein/cm² after three consecutive tape-strippings. In contrast, by day three of the experiment the MJ33 treated sites lost over 50 μg protein/cm² after three consecutive tape-strippings. These results show that two, chemically-unrelated secretory phospholipase A₂ inhibitors produce increases in skin surface pH, in parallel with diminished stratum corneum integrity and cohesion.

### Example 2

The experiments reported in this example show that the secretory phospholipase A₂ inhibitor induced decline in stratum corneum integrity and cohesion can be attributed to premature dissolution of desmosomes in the lower stratum corneum.

To further investigate the changes in stratum corneum cohesion that are brought about by application of secretory phospholipase A₂ inhibitors, BPB and MJ33 treated samples of murine skin and the corresponding untreated controls, were assessed by immunohistochemical staining and confocal microscopy. Both BPB and MJ33 provoked a dramatic reduction in the density of desmosomes in the lower stratum corneum and at the stratum corneum-stratum granulosum interface. In inhibitor treated sites, desmosomes were reduced in size and remnants of desmosomes were present at the stratum corneum-stratum granulosum interface. In vehicle treated sites, the number and appearance of desmosomes was normal.

A reduction in desmosomes was also seen by immunohistochemical assessment of desmoglein-1 (DSG1) positive structures in the lower stratum corneum of BPB vs. vehicle treated stratum corneum. On laser confocal microscopy, the density of DSG1 positive clusters, which are presumed to correspond to intact desmosomes, decline dramatically in BPB treated stratum corneum, while DSG1 positive staining in vehicle-treated stratum corneum is comparable to control.

These results show that the inhibitor induced decline in stratum corneum integrity and cohesion can be attributed to a premature dissolution of desmosomes in the lower stratum corneum.

### Example 3

The experiments reported in this example show that exposure of murine skin to neutral pH buffer induces functional alterations in the stratum corneum that mimic changes induced by secretory phospholipase A₂ inhibitors.

Stratum corneum integrity and cohesion were examined after short term exposure of normal skin to neutral (pH 7.4) vs. acidic (pH 5.5) pH. After 3 hours of exposure to a neutral pH buffer (HEPES, pH 7.4), the surface pH of the stratum corneum rose from 5.86+/−0.21 to 6.41+/−0.20. Exposure to an acidic buffer lowered the surface pH from 5.95+/−0.05 to 5.72+/−0.06. Whereas exposure to both buffers increased transepidermal water loss (TEWL) rates, a slightly greater increase in TEWL occurred following three hour exposure to the neutral pH buffer (all changes normal range).

Stratum corneum integrity was also reduced by exposure to neutral (pH 7.4) buffer. TEWL was measured after epidermal sites which had been exposed to either neutral or acidic buffers, as described in materials and methods, were insulted by a sequence of tape-strippings. After 4 tape-strippings, TEWL from sites exposed to neutral pH buffer occurred at a rate of over 75 g/m²/h. In contrast, the sites exposed to an acidic, pH 5.5 buffer, had TEWL levels close to 40 g/m²/h. The pattern persisted after five tape-strippings; here TEWL for the sites exposed to neutral buffer was about 90 g/m²/h and for sites exposed to pH 5.5 buffer TEWL was less than 75 g/m²/h (FIG. 3a).

Similarly, stratum corneum cohesion was impaired in skin treated with neutral pH buffer as compared to acidic pH buffer. Stratum corneum cohesion was measured as the amount of protein removed per D-Squame stripping. After five sequential strippings, the cumulative amount of protein removed was about 30 μg/cm² for skin treated with neutral pH buffer and less that 25 μg/cm² for skin exposed to acidic pH buffer. These studies show that short term exposure to neutral pH buffer produces functional abnormalities in stratum corneum integrity and cohesion which result in easier removal of the stratum corneum with tape stripping.

### Example 4

The experiments reported in this example demonstrate that exposure to an acidic pH buffer protects stratum corneum integrity from perturbation by inhibitors of secretory phospholipase A₂.

Murine skin that had been treated with BPB for three days, as described in materials and methods, was further exposed to either a neutral (pH 7.4) or acidic (pH 5.5) pH buffer for three hours. The increase in pH and abnormality in stratum corneum integrity provoked by the BPB treatment was accentuated by exposure of the BPB treated sites to neutral buffer for three hours. After the hairless mice were treated with secretory phospholipase A₂ inhibitor, BPB, for three days, the mice were further exposed to either a neutral or acidic pH buffer for three hours. Exposure to the neutral buffer (pH 7.4) amplified the pH increase induced by BPB treatment from about pH 5.95 to about pH 6.41. In contrast, exposure to the acidic buffer (pH 5.5) for three hours overrode and reversed the inhibitor induced pH increase; the starting pH for the sites exposed to acidic buffer was about pH 5.95 and after exposure to the acidic buffer for three hours, the surface pH was reduced to about pH5.8.

Stratum corneum barrier function as measured by rates of transepidermal water loss (TEWL), also revealed the beneficial effects of exposing the treated skin sites to an acidic pH buffer after treatment with BPB. Barrier function remained unchanged in BPB treated sites exposed for three hours to and acidic buffer, but skin sites exposed to a neutral buffer, showed an increase in TEWL from about 10 g/m²/h to about 18 g/m²/h.

Furthermore, stratum corneum integrity as measured by the rate of TEWL induced by sequential tape stripping, was reduced by exposure to the neutral buffer. After three sequential tape strippings of the BPB treated sites, sites that were further exposed to neutral buffer experienced TEWL rates of more than 60 g/m^{2/}h, whereas the sites that were exposed to acidic buffer experienced TEWL rates of less than 30g/m²/h.

The experiments reported in this example demonstrate that the abnormalities in stratum corneum pH, barrier function and integrity which are induced by secretory phospholipase A₂ inhibitor treatment, are enhanced by exposing the inhibitor treated sites to a neutral buffer, and can be ameliorated by the application of an acidic pH buffer.

### Example 5

The experiments reported in this example demonstrate that the end products of secretory phospholipase A₂ hydrolysis of phospholipids, free fatty acids (FFA), are responsible for maintaining the acidic environment of the stratum corneum and thereby, protect its integrity and cohesion. Thus, the application of substances that inhibit free fatty acid production will reduce cohesion and compromise integrity of the stratum corneum.

Hairless mice were treated with BPB or MJ33 alone and in combination with free fatty acid for three days as described on materials and methods. Following treatment, surface pH of the skin was measured. Co-application to hairless murine skin of the secretory phospholipase A₂ inhibitor BPB and palmitic acid (PA), stearic acid (SA) or to a lesser extent linoleic acid (LA), prevented the BPB induced increase in pH of the stratum corneum (PA: 5.40+/−0.14 vs. 5.92+/−0.05; SA: 5.73+/−0.09 vs. 6.17+/−0.08; LA: 5.77+/−0.10 vs. 6.28+/−0.1.). Furthermore, co-applications of BPB or MJ33 with palmitic acid, stearic acid and to a lesser extent linoleic acid also prevented emergence of secretory phospholipase A₂ inhibitor induced abnormalities in stratum corneum integrity and cohesion.

Mice were treated with BPB or MJ33 alone or in combination with free fatty acid for three days as noted above. Following treatment, the animals were tape stripped five times and the transepidermal water loss (TEWL) and cumulative protein removed per strip was measured for each of five successive tape strips. As expected, treatment of Murine skin with BPB or MJ33 alone produced significant and progressive increases in TEWL rates in response to each tape strip, indicating impaired integrity. Co-application of one of the fatty acids along with BPB or MJ33 protected stratum corneum integrity; TEWL progressively increased with increasing numbers of tape strips, but the increase was significantly less than the increase seen for BPB alone. By the fifth strip, TEWL levels for sites treated with BPB alone were between 80-100 g/m²/ h. Co-application of stearic acid with BPB held the TEWL levels to about 25 g/m²/h. Co-application of palmitic acid and BPB kept the TEWL levels down to about 55-60 g/m²/h. Similarly, after three sequential tape-strippings, sites treated with MJ33 alone had TEWL levels close to 75 g/m²/h. Co-application of palmitic acid with MJ33 kept TEWL levels down to about 30-35 g/m²/h.

Co-application of fatty acids and one or the other secretory phospholipase A₂ inhibitor also protected stratum corneum cohesion. The cumulative amount of protein removed by tape-stripping was significantly greater for the skin treated with BPB or MJ33 alone than it was for skin treated with BPB or MJ33 in combination with one of the fatty acids. After five sequential tape-strippings, sites treated with BPB alone lost about 125 μg protein/cm². Co-application of stearic acid kept the rate of protein loss per strip down to about 25 μg protein/cm². Although somewhat less effective than stearic acid, palmitic acid also facilitated the cohesion of the stratum corneum keeping the protein loss at the fifth strip down to about 40 μg/cm². Similarly, co-application of palmitic acid with MJ33 kept the loss of protein down to about 30 μg/cm² after three strippings, whereas the sites treated with MJ33 alone lost about 50 μg protein/cm² after three sequential strippings.

These results show that the secretory phospholipase A₂-inhibitor-induced abnormalities in integrity and cohesion of the stratum corneum are linked to increased pH, and that the presence of the end products of phospholipid hydrolysis, fatty acids, protect stratum corneum integrity and cohesion. Because free fatty acids are generated as an end product of phospholipid hydrolysis, and these free fatty acids acidify the stratum corneum, inhibitors of secretory phospholipase A₂ will increase the pH of the stratum corneum and thereby reduce integrity and cohesion.

### Example 6

The experiments reported in this example demonstrate that consistent with its role in maintaining the neutral intracellular pH of keratinocytes and the acidic pH of the stratum corneum extracellular domains, the NHE1 antiporter is located in cultured human keratinocytes (CHK) and also in the differentiated cell layers of epidermis.

The human isoform of NHE1 was demonstrated to be present in both cultured human keratinocytes (CHK) and in epidermis by RT-PCR. As shown in materials and methods, primers were chosen so that amplification of a 505 base pair band would be identify the human isoform of NHE 1. After isolation of mRNA from either epidermis or CHK as described in materials and methods, and RT-PCR, an RT-PCR product of the correct size (505 bp) and the correct sequence of that expected for the human isoform of NHE1 was generated.

NHE1 was also identified in preparations of CHK by western immunoblotting, as described in materials and methods, using an anti-NHE1 monoclonal antibody as a probe. The antibody identified a 114 kDa protein band, consistent with the predicted size of human NHE1.

Immunohistochemistry studies revealed NHE1 to be present in the outer nucleated layers of human epidermis, consistent with its proposed role in stratum corneum acidification. Human epidermal sections were stained with polyclonal NHE 1 antibody then detected with a n FITC-labeled secondary antibody as described in materials and methods. Tissue was counterstained with propidium iodide the visualized by confocal microscopy. Immunolabeling could be localized to the cytosol of suprabasal cells in epidermis. The same staining pattern was observed in NHE1 wild type (+/+) mice, but not in NHE1 knockout (−/−) mouse epidermis.

The immunohistochemical localization studies, combined with the evidence obtained from RT-PCR and western-blotting, demonstrate that NHE1 is expressed in differentiated cell layers and keratinocytes, consistent with its role in maintenance of intracellular pH and acidification of stratum corneum extracellular domains.

### Example 7

The experiments reported in this example demonstrate that the NHE1 antiporter is an important factor maintaining the intracellular pH in cultured human keratinocytes (CHK) and that NHE1 also contributes significantly to maintenance of an acid pH environment of the stratum corneum. The role of NHE1 in pH regulation in both CHK and the stratum corneum was investigated using two inhibitors specific for the NHE1 sodium proton exchanger, amiloride and HOE694.

First, activity of the NHE1 antiporter was tested by measuring the cells ability to recover from an acid load. As can be seen in FIG. 1, when NH₄Cl is added to cultures of CHK, the cells initially become more basic and, after dissociation of the ammonium ion, take on an acid load. Cells slowly extrude H⁺ re-establishing a more neutral pH. The addition of the specific NHE1 inhibitor, amiloride, to the culture at 1 μM, blocks the recovery of the cells from the acid load (FIG. 1). Consistent with this observation, when the NHE1 inhibitor HOE694 was applied at a concentration of 1.5 μM for 16 hours, the intracellular pH dropped from pH 7.05 to 6.90, suggesting that the cells are not able to pump out the extra H⁺ ions to normalize the intracellular pH (data not shown). These data show that NHE1 is present in human keratinocytes, and that NHE 1 activity regulates intracellular pH.

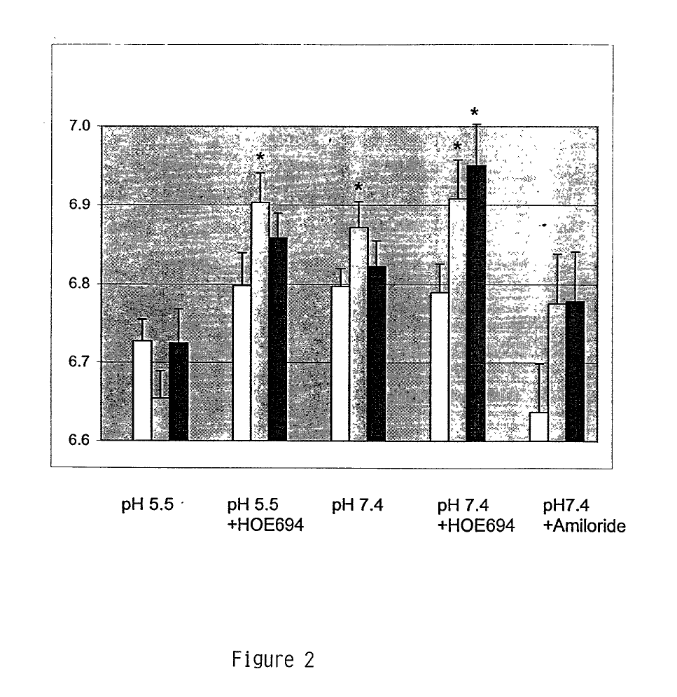

Next, investigation into the role played by NHE1 in maintaining the pH balance of the stratum corneum, was undertaken. The integrity of murine skin exposed to different buffers and NHE1 inhibitors was challenged by tape-stripping. The pH of the exposed skin was measured before and after tape-stripping. Before tape stripping, the surface pH in all animals was acidic (6.03+/−0.20, n=78). Tape stripping invariably resulted in an alkalinization of stratum corneum (6.77+/−0.15, n=156). Relative to the initial post-tape-strip time point, skin exposed to a HEPES based pH 5.5 buffer, became more acidic at 2 hrs post-tape-strip. At 5 hours post-tape strip the skin exposed to pH 5.5 buffer returned to its initial post tape strip value, suggesting that the stratum corneum had recovered from the initial acid load (FIG. 2). When the NHE1 inhibitor, HOE694, was added to the pH 5.5 buffer, the initial, post-tape-strip pH value was increased significantly with respect to the initial post-tape-strip value of skin exposed to pH 5.5 buffer alone. This result suggests that the mechanism responsible for acidifying the stratum corneum is inhibited. At two hours post-tape-strip, the skin exposed to HOE694 in pH 5.5 buffer was more alkaline than the initial post-tape-strip value by more than 0.1 pH units, strongly suggesting that the mechanism that acidifies the stratum corneum is blocked. At five hours post-tape-strip, these samples were still not fully recovered, remaining more than 0.05 pH units higher than the initial post-tape-strip values. Thus, these data suggest that the NHE1 antiporter has a significant effect in maintaining the acid environment of the stratum corneum.

Consistent with the results of the above described experiment, exposure of murine skin to pH 7.4 buffer resulted in a pH value at two hours post-tape strip that was increased by 0.075 pH units over the initial post-tape strip value. However, at five hours post tape strip, the pH of the skin exposed to pH 7.4 buffer was nearly recovered to its initial post-tape-strip value, suggesting that the NHE1 antiporter is still functioning. When the NHE1 inhibitor HOE694 was added to the pH 7.4 buffer, the initial post-tape-strip pH value was similar to the pH of the skin exposed to buffer only. However, at two hours post tape strip, the pH had increased significantly, being more that 0.1 pH unit higher than the initial post-tape-strip value and close to 0.05 pH units higher than the two hour time point of skin exposed to pH 7.4 buffer alone. Unlike the case for skin exposed to pH 7.4 buffer only, this elevated pH persisted and even increased at five hours post tape strip, suggesting that the mechanism that acidifies the stratum corneum is strongly blocked (FIG. 2).

These experiments demonstrate that the activity of the NHE1 antiporter is required in order to maintain the acidic pH of the normal stratum corneum. They also demonstrate that the activity of the NHE1 antiporter is stimulated by the application of an acid load, in the form of NH₄⁺ ions. As the N₄⁺ ion enters the cells of the outer epidermis, it is reduced to NH₃, which evaporates. The excess H+lowers the intracellular pH, activating the NHE1 antiporter to expel the excess H⁺. Expulsion of excess H⁺, in turn, leads to extracellular acidification of the outer epidermis and stratum corneum. Thus, NHE1 couples cellular physiology and the extracellular environment.

### Example 8

The experiments reported in this example demonstrate that NHE1 mediated acidification is linked to barrier function of the stratum corneum.

Transepidermal water loss (TEWL) was measured in order to assess the kinetics of barrier recovery following acute barrier perturbations induced by tape-stripping. Both of the NHE1 inhibitors, amiloride and HOE694, delayed barrier recovery.

Hairless mice were tape stripped to TEWL of 7-9 g/m^{2/}h. Hilltop Chambers with or without amiloride at 5 μM or HOE694 at 7.5 μM in 10 mM HEPES buffer adjusted to pH 7.4 or pH5.5 buffer were applied. Control areas were covered with Hilltop Chambers containing 10 mM HEPES buffer adjusted to pH 7.4 or pH 5.5 as appropriate. TEWL was measured at 0, 2, 5, and 24 hours post tape-strip. The results are reported as percent barrier recovery from the initial induced defect.

When either amiloride or HOE694 were applied in pH 7.4 buffer, barrier recovery was delayed relative to recovery of the skin treated with pH 7.4 buffer alone. At two hours post tape-strip, the skin treated with pH 7.4 buffer alone experienced a TEWL rate 21% less than the initial induced TEWL rate. In contrast, skin treated with HOE 694 or amiloride in pH 7.4 buffer had recovered to TEWL rates only about 12% less than the initial induced TEWL rate. Similarly, at five hours post tape-strip, the skin treated with pH 7.4 buffer had recovered to a TEWL rate about 38% less than the initial induced TEWL rate, while the skin treated with HOE 694 and pH 7.4 buffer had only recovered to a TEWL rate 23% less than the initial induced TEWL level and the skin treated with amiloride and pH 7.4 buffer had a TEWL rate only about 25% less than the initial rate induced by tape-stripping. The delay in barrier recovery was especially pronounced at 24 hours post tape-strip, at which point the skin treated with pH 7.4 buffer only, recovered to TEWL rates 75% less than the initial TEWL rate induced by tape-stripping. In contrast, skin treated with HOE 694 in pH 7.4 buffer recovered to TEWL levels only about 45% less than the initial induced TEWL rate.

Unlike the case of skin treated with HOE 694 in pH 7.4 buffer, skin treated with HOE694 in pH 5.5 buffer, recovered as well as the skin sites treated with pH 5.5 buffer only. A slight delay in barrier recovery was apparent at 2 hours post tape-strip, where skin sites treated with HOE 694 in pH 5.5 buffer had recovered to TEWL rates 25% less than initial rate induced by tape-stripping, whereas skin sites treated with pH 5.5 buffer alone recovered to TEWL rates 28% less than the initial induced rate. After the two hour time point, skin sites treated with HOE 694 in pH 5.5 buffer, as well as sites treated with pH 5.5 buffer and sites treated with pH 7.4 buffer recovered in parallel, experiencing TEWL rates 38% less than the initial induced value by 5 hours post tape-strip, and TEWL rates 75% less than the initial induced TEWL rate at 24 hours post tape-strip.

Thus, the pH of the applied buffer is less critical for barrier recovery when the NHE1 antiporter is functional. However, treatment of skin sites with pH 5.5 buffer facilitates barrier recovery in the presence of the NHE1 inhibitor. This suggests that the delay in barrier recovery seen in the skin treated with HOE 694 in pH 7.4 buffer is due to an alteration in stratum corneum pH which, in the absence of NHE1 function, can be manipulated by application of buffers of different pH values.

### Example 9

The experiments reported in this example demonstrate that permeability barrier homeostasis is abnormal in transgenic NHE1 knockout mice.

NHE1 knockout mice were generated via gene targeting to eliminate NHE1. Barrier function was first assessed under basal conditions and neither the surface pH nor the baseline transepidermal water loss (TEWL) rates differed significantly between NHE −/− and +/+ littermates. However, after the integrity of the epidermal barrier was challenged by tape-stripping, differences in barrier recovery became apparent. Barrier recovery was significantly delayed in −/− mice compared with their wild type littermates, especially at 5 and 8 hours post tape-strip.

Paired mice were shaved and tape-stripped as in Example 8 above. Barrier recovery was then measured as percent recovery from the intial defect. At five hours post tape strip, barrier function in wild type (+/+) mice had recovered to TEWL rates that were 30% less than the initial defect, whereas their −/− littermates recovered to TEWL rates that were only 20% less than the initial TEWL rates induced by tape-stripping. Similarly, at 8 hours post tape-strip, wild type mice had recovered to TEWL rates that were 45% less than the TEWL rate induced by tape-stripping, whereas their −/− litternates recovered to TEWL rates that were only 30% less than the initial defect. The pattern of recovery resembled that seen with the topical inhibitor HOE694 in pH 7.4 buffer described in Example 8 above.

Thus, these results demonstrate that the NHE1 antiporter is necessary for normal recovery of barrier function following disruption by tape-stripping.

### Example 10

The experiments reported in this example demonstrate that the inhibition of NHE1 activity affects extracellular processing of secreted stratum corneum lipids.

The mechanistic basis for the inhibitor and NHE 1 knockout induced delays in barrier recovery (described in Examples 8 and 9, respectively) was investigated using electron microscopy. Two competing hypotheses were tested. The delay in barrier recovery could be due to defects in lamellar body formation and secretion of stratum corneum lipids or instead, could be due to extracellular processing of stratum corneum lipids following secretion.

Biopsies were taken during time course experiments. When EM images from animals treated with HOE694 in pH 5.5 buffer were viewed under the electron microscope, both lamellar body formation and extracellular processing of secreted lipids appeared normal. Interestingly, when EM images from animals treated with HOE694 in a pH 7.4 buffer were assessed, it was found that lamellar body formation and secretion were undisturbed, however, the persistence of incomplete, immature extracellular lamellar bilayer structures suggested that extracellular processing of secreted lipids was defective. These results suggest that inhibition of NHE1 activity interferes with extracellular processing of secreted stratum corneum lipids, and that this effect is enhanced by the application of a neutral (pH 7.4) buffer.

Similarly, electron micrographs of biopsies taken from NHE1 −/− knockout mice revealed the persistence of newly secreted lipids several layers above the stratum corneum-stratum granulosum interface, and the presence of incompletely processed lamellar membrane structures. Thus, extracellular lipid processing appears to be delayed in NHE1 knockout (−/−) mice in the same way that it is in mice that have been treated with the NHE1 inhibitor, HOE694, in neutral buffer.

These results show that the skin of NHE1 knockout mice and the skin of mice treated with the NHE1 inhibitor, HOE694, in pH 7.4 buffer both have similar defects in extracellular processing of secreted stratum corneum lipids. Processing of secreted lipids results in the production of free fatty acids which acidify the stratum corneum and protect its integrity and cohesion (see Example 1). Thus, application of NHE1 inhibitors will reduce stratum corneum integrity and cohesion and thereby make it easier to detach layers of stratum corneum as necessary.

### Example 11

The experiments reported in this example show that even limited application of neutral pH buffer results in decreased cohesion of the stratum corneum of human feet.

The soles of the feet of ten human subjects were treated with HEPES buffer adjusted to neutral pH (pH 7.4) or acidic pH (pH 5.5). Soaked discs of filter paper (Whatman 1, Whatman Int. Ltd., Maidstone England) with a diameter of 1 cm were placed on both heels of each subject and fixed with Tegaderm (3M, St Paul Minn.). After three hours the discs were removed. Twenty minutes later ten sequential tape-strippings with D-Squame tapes were taken from both treated sites. The cumulative amount of protein removed was then measured as described in Materials and Methods.

After ten strippings, the sites exposed to the pH 7.4 buffer had lost on average about 500 μg protein/cm², whereas the sites exposed to the pH 5.5 buffer lost on average less than 375 μg protein/cm². Thus, treatment with a neutral pH buffer facilitated the removal of normal stratum corneum.

The results of these experiments demonstrate that the application of a neutral pH buffer reduces stratum corneum cohesion thereby allowing for easier removal of the stratum corneum by tape stripping.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for treating the epidermis of a terrestrial mammalian subject suffering from hyperkeratosis, visible scale or both due to increased intercellular cohesion of the stratum corneum, said method comprising administering to said epidermis a topical composition comprising an active ingredient that neutralizes the pH of the stratum corneum and is a member selected from the group consisting of inhibitors of proton pumps, transporters or antiporters, inhibitors of free fatty acid generation from complex lipids, inhibitors of proteolytic processes that generate organic acids and buffers with a pH of 7.0 or greater, said active ingredient being present in a concentration that is effective in raising the pH of said epidermis thereby reducing intercellular cohesion and resulting in easier detachment of normal stratum corneum and easier removal of hyperkeratotic stratum corneum.

2. A method in accordance with claim 1 in which said active ingredient is an inhibitor of proton pumps, transporters or antiporters.

3. A method in accordance with claim 2 in which said inhibitor is a an inhibitor of NHE1.

4. A method in accordance with claim 2 in which said inhibitor is a a member selected from the group consisting of amiloride, dimethylamiloride, ethylisopropylamiloride, methylpropylamiloride, methylisobutylamiloride, hexametyleneamiloride, HOE694 and HOE642.

5. A method in accordance with claim 2 in which said inhibitor is amiloride.

6. A method in accordance with claim 2 in which said inhibitor is HOE694.

7. A method in accordance with claim 1 in which said active ingredient is an inhibitor of free fatty acid generation from complex lipids.

8. A method in accordance with claim 7 in which said inhibitor is an inhibitor of phospholipase A₂.

9. A method in accordance with claim 7 in which said inhibitor is a member selected from the group consisting of bromphenacyl bromide, 1-hexadecyl-3-trifluoroethylglycero-sn-2-phosphomethanol, quinacrine and mepacrine.

10. A method in accordance with claim 7 in which said inhibitor is bromphenacyl bromide.

11. A method in accordance with claim 7 in which said inhibitor is 1-hexadecyl-3-trifluoroethylglycero-sn-2-phosphomethanol.

12. A method in accordance with claim 7 in which said inhibitor is an inhibitor of β-glucocerebrosidase.

13. A method in accordance with claim 7 in which said inhibitor is a member selected from the group consisting of bromconduritol-β-epoxide.

14. A method in accordance with claim 1 in which said active ingredient is an inhibitor of proteolytic processes that lead to the generation organic acids.

15. A method in accordance with claim 14 in which said inhibitor is an inhibitor which blocks proteolytic processes that breakdown filaggrin.

16. A method in accordance with claim 1 in which said inhibitor is an inhibitor which blocks processes that generate urocanic acid.

17. A method in accordance with claim 16 in which said inhibitor is a member selected from the group consisting of histidase inhibitors.

18. A method in accordance with claim 16 in which said inhibitor is nitromethane or histidinol phosphate.

19. A method in accordance with claim 1 in which said active ingredient is a buffer solution with a pH 7.0 or greater.

20. A method in accordance with claim 19 in which said buffer solution is a member selected from the group consisting of solutions of HEPES-based, MES-based, MOPS-based, PIPES-based, TES-based, phosphate-based, citrate-based or bicarbonate-based buffers.

21. A method in accordance with claim 19 in which said buffer solution is a solution of a HEPES-based buffer.

22. A method in accordance with claim 19 in which said buffer solution is a solution with a pH range of 7.0-8.0.

23. A method in accordance with claim 19 in which said buffer solution is a pH 7.4 buffer.

24. A method in accordance with claim 1 in which said topical composition comprises two or more of said active ingredients.

25. A method for manipulating the epidermis of a terrestrial mammalian subject with the purpose of providing improved transdermal delivery of co- or sequentially applied drugs or nutrients for systemic, regional or local applications, said method comprising administering to said epidermis a topical composition comprising an active ingredient that neutralizes the pH of the stratum corneum and is a member selected from the group consisting of inhibitors of proton pumps, transporters or antiporters, inhibitors of free fatty acid generation from complex lipids, inhibitors of proteolytic processes that generate organic acids, and buffers with a pH of 7.0 or greater, said active ingredient being present in a concentration that is effective at raising the pH of said epidermis with the purpose of reducing intercellular cohesion and integrity of the stratum corneum to breakdown the epidermal permeability barrier.

26. A method in accordance with claim 25 in which said active ingredient is an inhibitor of proton pumps, transporters or antiporters.

27. A method in accordance with claim 26 in which said inhibitor is a an inhibitor of NHE1.

28. A method in accordance with claim 26 in which said inhibitor is a a member selected from the group consisting of amiloride, dimethylamiloride, ethylisopropylamiloride, methylpropylamiloride, methylisobutylamiloride, hexametyleneamiloride, HOE694 and HOE642.

29. A method in accordance with claim 26 in which said inhibitor is amiloride.

30. A method in accordance with claim 26 in which said inhibitor is HOE694.

31. A method in accordance with claim 25 in which said active ingredient is an inhibitor of free fatty acid generation from complex lipids.

32. A method in accordance with claim 31 in which said inhibitor is an inhibitor of phospholipase A₂.

33. A method in accordance with claim 31 in which said inhibitor is a member selected from the group consisting of bromphenacyl bromide, 1-hexadecyl-3-trifluoroethylglycero-sn-2-phosphomethanol, quinacrine and mepacrine.

34. A method in accordance with claim 31 in which said inhibitor is bromphenacyl bromide.

35. A method in accordance with claim 31 in which said inhibitor is one hexadecyl-3-trifluoroethylglycero-sn-2-phosphomethanol.

36. A method in accordance with claim 31 in which said inhibitor is an inhibitor of β-glucocerebrosidase.

37. A method in accordance with claim 31 in which said inhibitor is a member selected from the group consisting of bromconduritol-β-epoxide.

38. A method in accordance with claim 25 in which said active ingredient is an inhibitor of proteolytic processes that lead to the generation organic acids.

39. A method in accordance with claim 38 in which said inhibitor is an inhibitor which blocks proteolytic processes that break down filaggrin.

40. A method in accordance with claim 25 in which said inhibitor is an inhibitor which blocks processes that generate urocanic acid.

41. A method in accordance with claim 40 in which said inhibitor is a member selected from the group consisting of histidase inhibitors.

42. A method in accordance with claim 40 in which said inhibitor is nitromethane or histidinol phosphate.

43. A method in accordance with claim 25 in which said active ingredient is a buffer solution with a pH 7.0 or greater.

44. A method in accordance with claim 43 in which said buffer solution is a member selected from the group consisting of solutions of HEPES-based, MES-based, MOPS-based, PIPES-based, TES-based, phosphate-based, citrate-based or bicarbonate-based buffers.

45. A method in accordance with claim 43 in which said buffer solution is a solution of a HEPES-based buffer.

46. A method in accordance with claim 43 in which said buffer solution is a solution with a pH range of 7.0-8.0.

47. A method in accordance with claim 43 in which said buffer solution is a pH 7.4 buffer.

48. A method in accordance with claim 25 in which said topical composition comprises two or more of said active ingredients
